(19) United States
(12) Patent Application Publication
(10) Pub. No.: US 2002/0140903 A1
(43) Pub. Date: Oct. 3, 2002

(54) System and method for providing an improved test for determining the resolving power of the eye

(75) Inventor: Ronald A. Schachar, Dallas, TX (US)

Correspondence Address: Docket Clerk, P.O Drawer 800889, Dallas, TX, 75380, US

(73) Assignee: RAS Holding Corp, Dallas, TX

(21) Appl. No.: 10/108,918

(22) Filed: Mar. 28, 2002

Related U.S. Application Data
(60) Provisional application No. 60/279,537, filed on Mar. 28, 2001.

(51) Int. Cl. A61B 3/10

(52) U.S. Cl. 351/221

(57) ABSTRACT

A system and method is disclosed for determining the resolving power of an eye. An eye is simultaneously illuminated with a first colored light source and a second colored light source. The colored light sources are moved toward the eye. At a point where the eye ceases to resolve the images of the two light sources the two colors of the light sources combine to form a third color. The eye easily perceives the sudden appearance of the third color that indicates the loss of resolution. In an alternate embodiment a display screen provides two colored light sources that flicker at different flicker rates. By selecting certain values for the flicker rates the two colored light sources combine to form a light that does not appear to flicker at the point where the eye ceases to resolve the images of the two colored flickering light sources.

### PRIORITY CLAIM TO PROVISIONAL PATENT APPLICATION

This patent application claims priority to U.S. Provisional Patent Application Serial No. 60/279,537 filed on Mar. 28, 2001.

### TECHNICAL FIELD OF THE INVENTION

The present invention is generally directed to systems and methods for testing and evaluating vision, and, in particular, to systems and methods for testing and determining the resolving power of the eye.

### BACKGROUND OF THE INVENTION

The structure and optical functions of the eye have been known for centuries. As early as 1619, the general principles of the operation of the eye were described by Christoph Scheiner (1575-1650) in his work “Oculus, hoc est: fundamentum opticum.” As is well known, the lens of the eye focuses light rays on the retina of the eye. The optical nerve carries electrical signals from the retina to the brain. The optical processing center of the brain interprets the electrical signals as images.

The term “resolution” in optics refers to the ability of an optical system (including the eye) to individually perceive two (or more) separate light sources when the light sources are located close to each other. The eye is said to “resolve” two (or more) images when the images are individually discernible.

A number of methods exist for testing and determining the resolving power of the eye. One well known method involves having a person view light from two light sources that are located close together. It is common practice for the two light sources to be formed by making two parallel slits in an opaque screen. As a light source illuminates the back of the screen, the two parallel slits in the screen pass two slits of light through the screen. Viewing the front of the screen, a person sees an image of two slit shaped light sources. Although light sources having other shapes may be used, for convenience of explanation, the two light sources will hereafter be assumed to be two slit shaped light sources.

The resolving power of the eye may be tested by causing the screen to be moved along an optical axis that passes through the center of the lens of the eye that is being tested. At a range of locations along the optical axis, the eye is capable of focusing light rays from each of the two light sources. When the light rays from each of the two slit shaped light sources are focused, the person being tested perceives the two images as two distinct and separate images of slits of light. The two images are said to be “resolved” by the eye.

As the screen is moved closer to the eye, at some point (which varies from individual to individual) the lens of the eye is no longer able to separately focus the two images on the retina of the eye. When this happens, the light rays from the two slit shaped light sources begin to overlap on the same area of the retina of the eye. The person then perceives a merging and blurring of the boundaries of the slit shaped light sources. The images are then said to be “not resolved” by the eye.

The distance from the eye at which the ability to resolve the two slit shaped light sources is lost provides a measure of the resolving power of the eye. In order to obtain an accurate measure of the resolving power of the eye, it is important to be able to accurately measure the location of the point on the optical axis when the eye ceases to be able to resolve the two images.

In practice, the person whose eye is being tested must make a subjective determination as to when he or she is no longer able to resolve the two images. As the screen with the two light sources is moved closer and closer to the eye, it is not always easy for a person to determine exactly when the eye ceases to resolve the two images. It is not always clear exactly when the transition from “resolution” to “no resolution” occurs.

Another prior art method provides an alternative to using a light source and a screen with two slits. This alternative method uses an eye chart having two parallel black lines located close together. At a range of locations along the optical axis, the eye is capable of focusing light rays from each of the two black lines. When the light rays from each of the two black lines are focused, the person being tested perceives the two images as two distinct and separate images of black lines. The images of the two black lines are said to be “resolved” by the eye.

As the eye chart is moved closer to the eye, at some point (which varies from individual to individual) the lens of the eye is no longer able to separately focus the two images of the black lines on the retina of the eye. When this happens, the light rays from the two black lines begin to overlap on the same area of the retina of the eye. The person then perceives an image of three parallel black lines. The image of the third black line is located between the images of the two original black lines. The eye perceives the image of the third black line due to the overlapping light rays of the two original black lines. The images of the two original black lines are then said to be “not resolved” by the eye.

The person whose eye is being tested must make a subjective determination as to when he or she is no longer able to resolve the images of the two black lines. As the eye chart with the two black lines is moved closer and closer to the eye, it is not always easy for a person to determine exactly when the eye ceases to resolve the two images. That is, the person may have difficulty determining exactly when the two images begin to become unfocused, thereby causing an image of a third black line to appear. In this alternative prior art method, it is not always clear exactly when the transition from “resolution” to “no resolution” occurs.

Therefore, there is a need in the art for a system and method that will provide an improved test for determining the resolving power of the eye. In particular, there is a need in the art for a system and method that will provide an improved test that will make it easier for a person to have an easily definable end point to determine when his or her eye is no longer able to resolve two (or more) visual images.

### SUMMARY OF THE INVENTION

The present invention generally comprises an improved system and method for providing an improved test for determining the resolving power of the eye.

In an advantageous embodiment of the present invention, the invention uses a first light source having a first color and a second light source having a second color. When the eye resolves the two light source images, the eye perceives a first image in the first color and a second image in the second color. When the eye is not able to resolve the two light source images, the eye perceives an image having a color that is an additive mixture of the first color and the second color. The point at which the eye changes from the state of “resolution” of the two images to the state of “no resolution” of the two images is very noticeable because of the sudden appearance of the new color that occurs as a result of the additive mixture of the first color and the second color.

In an advantageous embodiment of the present invention, the improved method of the invention comprises the steps of (1) illuminating the lens of an eye with a first light source having a first color, and (2) simultaneously illuminating the lens of the eye with a second light source having a second color, and (3) moving the first light source and the second light source toward the eye until the eye is no longer able to resolve the images of the two light sources, and (4) determining the point at which the eye is no longer able to resolve the images of the two light sources by identifying the point where the first light source color and the second light source color combine to form a third color that is an additive mixture of the first and second colors.

In another advantageous embodiment of the present invention, the invention uses an eye chart having a first colored line and a second colored line. When the eye resolves the images of the colored lines, the eye perceives a first image of a line having a first color and a second image of a line having a second color. When the eye is not able to resolve the two images of the two colored lines, the eye perceives an image of a line having a color that is an additive mixture of the first color and the second color. The point at which the eye changes from the state of “resolution” of the images of the two colored lines to the state of “no resolution” of the images of the two colored lines is very noticeable because of the sudden appearance of a line having a new color that occurs as a result of the additive mixture of the first color and the second color.

In another advantageous embodiment of the present invention, the improved method of the invention comprises the steps of (1) illuminating the lens of an eye with light from a first colored line, and (2) simultaneously illuminating the lens of the eye with light from a second colored line, and (3) moving the first colored line and the second colored line toward the eye until the eye is no longer able to resolve the images of the two colored lines, and (4) determining the point at which the eye is no longer able to resolve the images of the two colored lines by identifying the point where the color of the first colored line and the color of the second colored line combine to form a line having a third color that is an additive mixture of the first and second colors.

In another advantageous embodiment of the present invention, the invention uses a display screen (e.g., a computer monitor) that is capable of displaying flickering colored lines. The display screen displays a first colored line that flickers at a first flicker rate and a second colored line that flickers at a second flicker rate. By selecting certain values for the first and second flicker rates, it is possible to create a line having a third color that is an additive mixture of the first and second colors, where the central line does not appear to flicker. The point at which the eye changes from the state of “resolution” of the images of the two colored flickering lines to the state of “no resolution” of the images of the two colored flickering lines is very noticeable because of the sudden appearance of a non-flickering line having a new color that occurs as a result of the additive mixture of the first color and the second color.

Alternatively, by selecting other values for the first and second flicker rates, it is possible to create a line having a third color that is an additive mixture of the first and second colors, where the central line does appear to flicker, and the flickering of the first and second colored lines is not apparent to the eye. The point at which the eye changes from the state of “resolution” of the images of the two colored apparently non-flickering lines to the state of “no resolution” of the images of the two colored apparently non-flickering lines is very noticeable because of the sudden appearance between them of a flickering line having a new color that occurs as a result of the additive mixture of the first color and the second color.

It is a primary object of the present invention to provide an improved test to determine the point at which an eye is no longer able to resolve the images of two light sources.

It is another object of the present invention to provide an improved test to determine the point at which an eye is no longer able to resolve the images of two lines.

It is another object of the present invention to provide an improved test for determining the resolving power of the eye using a first color light source and a second color light source.

It is another object of the present invention to provide an improved test for determining the resolving power of the eye using a first colored line on an eye chart and a second colored line on an eye chart.

It is an additional object of the present invention to provide an improved test for determining the resolving power of an eye by causing the light from a first color light source and the light from a second color light source to combine to form a third color when the eye is not able to resolve the images of the first color light source and of the second color light source.

It is yet another object of the present invention to provide an improved test for determining the resolving power of an eye using by causing the light from a first colored line and the light from a second colored line to combine to form a third color when the eye is not able to resolve the images of the first colored line and of the second colored line.

It is another object of the present invention to provide an improved test for determining the resolving power of the eye using a first color light source that flickers at a first flicker rate and a second color light source that flickers at a second flicker rate.

It is another object of the present invention to provide an improved test for determining the resolving power of the eye using a light source that flickers at a first flicker rate and a second light source that flickers at a second flicker rate, where light from the two light sources combines to form an image that does not appear to flicker when the two light sources are not resolved.

It is another object of the present invention to provide an improved test for determining the resolving power of the eye using a light source that flickers at a first flicker rate and a second light source that flickers at a second flicker rate, where light from the two light sources combines to form an image that does appear to flicker when the two light sources are not resolved.

The foregoing has outlined rather broadly the features and technical advantages of the present invention so that those skilled in the art may better understand the Detailed Description of the Invention that follows. Additional features and advantages of the invention will be described hereinafter that form the subject of the claims of the invention. Those skilled in the art should appreciate that they may readily use the conception and the specific embodiment disclosed as a basis for modifying or designing other structures for carrying out the same purposes of the present invention. Those skilled in the art should also realize that such equivalent constructions do not depart from the spirit and scope of the invention in its broadest form.

Before undertaking the Detailed Description of the Invention, it may be advantageous to set forth definitions of certain words and phrases used throughout this patent document: the terms “include” and “comprise” and derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller,” “processor,” or “apparatus” means any device, system or part thereof that controls at least one operation, such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document, those of ordinary skill in the art should understand that in many, if not most instances, such definitions apply to prior uses, as well as to future uses, of such defined words and phrases.

### BRIEF DESCRIPTION OF THE DRAWINGS

For a more complete understanding of the present invention, and the advantages thereof, reference is now made to the following descriptions taken in conjunction with the accompanying drawings, wherein like numbers designate like objects, and in which:

FIG. 1 is a diagram illustrating how light from a light source passes through two slits in an opaque screen to form two light source images for testing the resolving power of an eye;

FIG. 2 is a diagram illustrating exemplary dimensions of two parallel slits in an opaque screen of the type shown in FIG. 1;

FIG. 3 is a diagram illustrating how two white light images are resolved on the retina of an eye when the light source images are sufficiently far from the lens of the eye;

FIG. 4 is a diagram illustrating how two white light images are not resolved on the retina of an eye when the light source images are close to the lens of the eye;

FIG. 5 is a diagram illustrating how a yellow light image and a blue light image of the present invention are resolved on the retina of an eye when the yellow and blue light source images are sufficiently far from the lens of the eye;

FIG. 6 is a diagram illustrating how a yellow light image and a blue light image of the present invention are not resolved on the retina of an eye when the yellow and blue light source images are close to the lens of the eye;

FIG. 7 is a diagram illustrating how a red light image and a green light image of the present invention are not resolved on the retina of an eye when the red and green light source images are close to the lens of the eye;

FIG. 8 is a diagram illustrating a prior art eye chart showing two black parallel lines;

FIG. 9 is a diagram illustrating an eye chart of the present invention showing two colored parallel lines, each colored with a different color;

FIG. 10 is a diagram illustrating a display screen of the present invention displaying two colored parallel lines, each colored with a different color;

FIG. 11 is a diagram illustrating a display screen of the present invention displaying two colored parallel lines, where each colored line flickers with a different frequency;

FIG. 12 is a diagram illustrating the appearance of a central line that is an additive mixture of a first color that flickers with a first frequency and a second color that flickers with a second frequency, where the central line does not appear to flicker; and

FIG. 13 is a diagram illustrating the appearance of a central line that is an additive mixture of a first color that flickers with a first frequency and a second color that flickers with a second frequency, where the central line does appear to flicker.

### DETAILED DESCRIPTION OF THE INVENTION

FIGS. 1 through 13, discussed below, and the various embodiments set forth in this patent document to describe the principles of the improved system and method of the present invention are by way of illustration only and should not be construed in any way to limit the scope of the invention. Those skilled in the art will readily understand that the principles of the present invention may also be successfully applied in any similar type of device for determining the resolving power of an eye. It is noted that the illustrations shown in FIGS. 1 through 7 are not drawn to scale.

FIG. 1 illustrates a prior art system that is used to determine the resolving power of an eye. White light from light source 110 passes through a first slit 120 and through a second slit 130 in opaque screen 140. Eye 150 perceives a first image that is due to the light from slit 120 in screen 140. Eye 150 also perceives a second image that is due to the light from slit 130 in screen 140. As previously mentioned, when screen 140 is sufficiently far from eye 150, eye 150 is able to resolve (i.e., individually distinguish) the two images from slit 120 and slit 130.

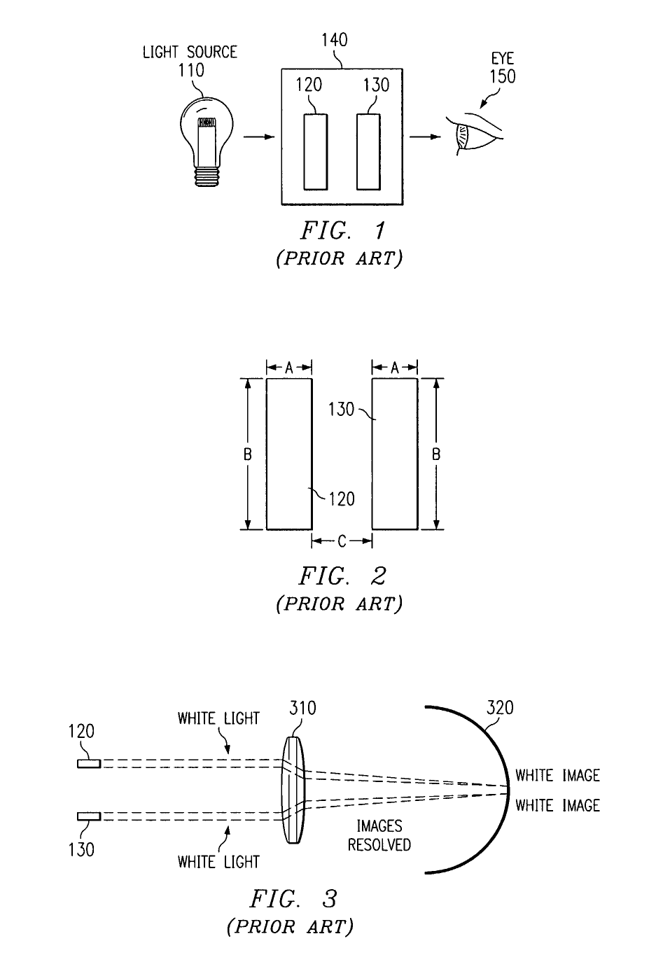

FIG. 2 illustrates exemplary dimensions of slit 120 and slit 130 in screen 140. An exemplary width for slit 120 and for slit 130 is designated with the letter “A” in FIG. 2. The width is approximately two hundred (200) microns. A micron is equal to one millionth of a meter. A micron is therefore equal to one thousandth of a millimeter. This means that two hundred microns is 0.20 of a millimeter. This distance is approximately one fifth of a millimeter. The dimensions of slit 120 and slit 130 are not drawn to scale in FIG. 2.

An exemplary height for slit 120 and for slit 130 is designated with the letter “B” in FIG. 2. The height may be selected from a range of length values. In particular, the height may be from five (5) millimeters to ten (10) millimeters. An exemplary dimension for the separation between slit 120 and slit 130 is designated with the letter “C” is FIG. 2. The separation distance may be selected from a range of length values. In particular, the separation (from the edge of slit 120 to the edge of slit 130) may be from one hundred (100) microns (or 0.10 millimeter) to one thousand (1000) microns (or 1.0 millimeter). An optimal value for the separation distance is three hundred (300) microns (or 0.30 millimeter) to five hundred (500) microns (or 0.50 millimeter).

FIG. 3 is a diagram illustrating a prior art system in which a white light image from slit 120 and a white light image from slit 130 are resolved on the retina 320 of eye 150 when screen 140 is sufficiently far from lens 310 of eye 150. Lens 310 focuses the white light image from slit 120 on retina 320 at a first location. Lens 310 also focuses the white light image from slit 130 on retina 320 at a second location. Eye 150 perceives two separate and distinct white light images in the form of two white vertical lines separated by a black line. The black line between the two white vertical lines is due to the portion of opaque screen 140 between slit 120 and slit 130.

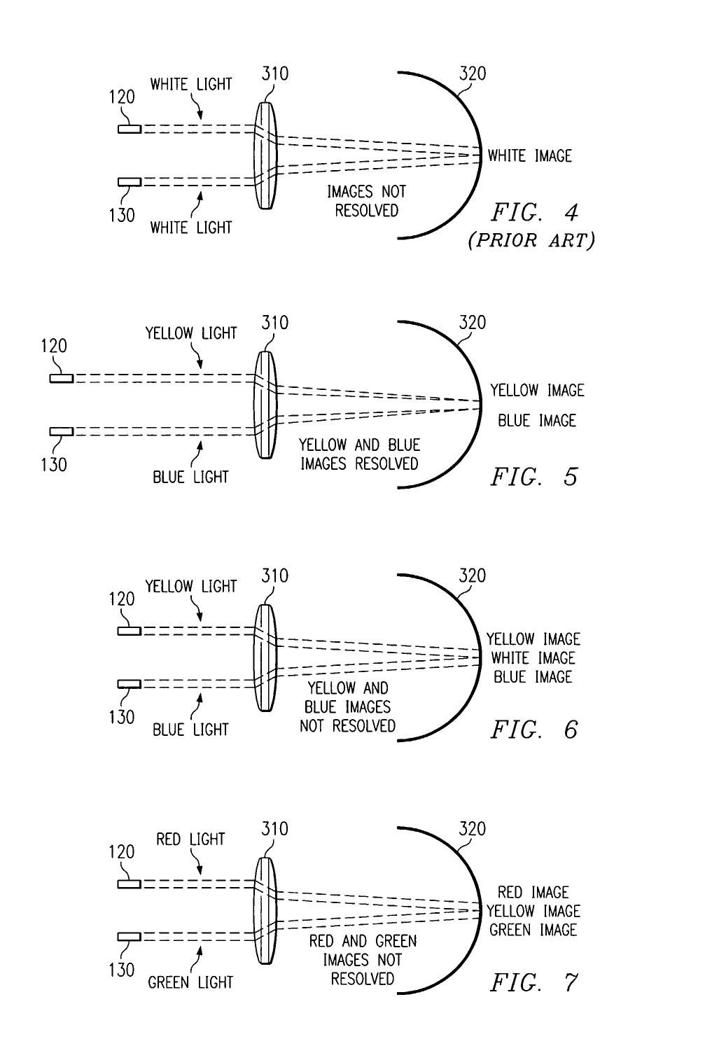

FIG. 4 is a diagram illustrating the prior art system shown in FIG. 3 in which the distance from lens 310 of eye 150 to screen 140 has been significantly shortened compared with the distance shown in FIG. 3. In this situation the white light image from slit 120 and the white light image from slit 130 are not resolved on the retina 320 of eye 150. The overlapping of the two white light images creates a single white light image on retina 320. In order to determine when the two white light images are no longer resolved, the person whose eye is being testing must sense when the black portion between the two white light images disappears. Because the two white light images merge into one white light image as they overlap, the black portion disappears gradually. Therefore it is not easy to determine when the black portion has completely disappeared.

In contrast, the system of the present invention uses a first color light source for slit 120 and a second color light source for slit 130. In the exemplary embodiment shown in FIG. 5, the color of the light from slit 120 is yellow and the color of the light from slit 130 is blue. This selection of colors is exemplary only. That is, the present invention is not limited to use of the colors yellow and blue. It is clear that other suitable combinations of colors may be used. The principle of operation of the present invention will work equally well with other suitable combinations of colors.

FIG. 5 is a diagram illustrating the system of the present invention in which a yellow light image from slit 120 and a blue light image from slit 130 are resolved on the retina 320 of eye 150 when screen 140 is sufficiently far from lens 310 of eye 150. Lens 310 focuses the yellow light image from slit 120 on retina 320 at a first location. Lens 310 also focuses the blue light image from slit 130 on retina 320 at a second location. Eye 150 perceives two separate and distinct images in the form of a yellow vertical line and a blue vertical line separated by a black line. The black line between the yellow and blue vertical lines is due to the portion of opaque screen 140 between slit 120 and slit 130.

FIG. 6 is a diagram illustrating the system of the present invention shown in FIG. 5 in which screen 140 is closer to eye 150. Either screen 140 is moved closer to eye 150 or eye 150 is moved closer to screen 140. The distance from lens 310 of eye 150 to screen 140 has been significantly shortened compared with the distance shown in FIG. 5. In this situation the yellow light image from slit 120 and the blue light image from slit 130 are not resolved on the retina 320 of eye 150. However, the overlapping of the yellow light image and the blue light image creates a white light image on retina 320. This is due to the fact that the color of white is an additive mixture of the colors yellow and blue.

In order to determine when the yellow light image and the blue light image are no longer resolved, the person whose eye is being tested can easily sense when the black portion between the yellow light image and the blue light image has changed to a white light image. Because the yellow light image and the blue light image merge into a white light image as they overlap, it is easily determined when the black portion has disappeared and been replaced with a white light image. This is because the change from black to white is very sudden and very noticeable. The suddenness of the change from black to white is more noticeable than the appearance of a black line between two black line images as described in the prior art. The image contrast provided by the present invention is noticeably greater than the image contrast provided by prior art systems.

As previously mentioned, the use of a yellow light image and a blue light image is only one example of the colors that may be used in the present invention. It is clear that other suitable color combinations may be used in other advantageous embodiments of the invention. For example, as shown in FIG. 7, light from red and green illuminated slits will additively mix to yield a yellow line. In this situation the red light image from slit 120 and the green light image from slit 130 are not resolved on the retina 320 of eye 150. However, the overlapping of the red light image and the green light image creates a yellow light image on retina 320. This is due to the fact that the color of yellow is an additive mixture of the colors red and green.

However, some persons are color blind and are not able to distinguish the colors of red and green. To test color blind persons, it may be necessary to use two other colors that combine to produce an additive mixture that is readily discernible.

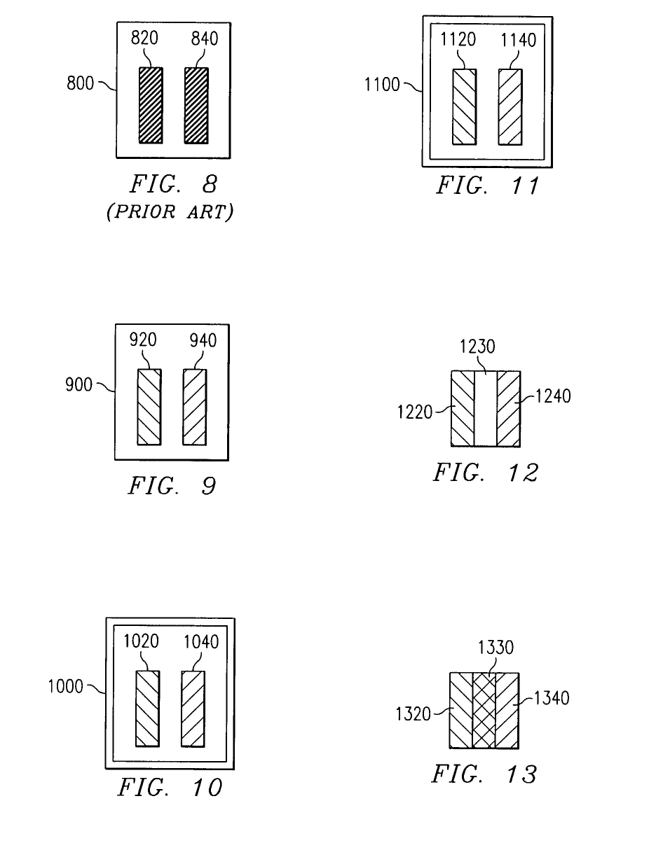

In an alternate embodiment of the invention, an eye chart may be used in place of screen 140. FIG. 8 is a diagram illustrating prior art eye chart 800. Eye chart 800 has two black parallel lines, 820 and 840, located close together. At a range of locations along the optical axis, the eye is capable of focusing light rays from each of the two black lines, 820 and 840. When the light rays from each of the two black lines, 820 and 840, are focused, the person being tested perceives the two images as two distinct and separate images of black lines. The images of the two black lines are said to be “resolved” by the eye.

As eye chart 800 is moved closer to eye 150, at some point (which varies from individual to individual) the lens 310 of eye 150 is no longer able to separately focus the two images of the black lines, 820 and 840, on the retina 320 of eye 150. When this happens, the light rays from the two black lines, 820 and 840, begin to overlap on the same area of the retina 320 of eye 150. The person then perceives an image of three parallel black lines. The image of the third black line is located between the images of the two original black lines, 820 and 840. Eye 150 perceives the image of the third black line due to the overlapping light rays of the two black lines. The images of the two black lines, 820 and 840, are then said to be “not resolved” by the eye.

FIG. 9 is a diagram illustrating eye chart 900 of the present invention. Eye chart 900 has two colored parallel lines, 920 and 940. Line 920 and line 940 are each colored with a different color. Line 920 and line 940 are located with respect to each other in the same fashion as slit 120 and slit 130. In this embodiment of the invention, a direct light source 110 is not used. Instead, eye 150 perceives the colors of the colored lines on eye chart 900 from reflected ambient light. The principles of operation of this alternate embodiment using eye chart 900 are the same as the principles of operation that have been previously described for the case of two separate light sources.

In particular, when eye 150 resolves the image of colored line 920 and the image of colored line 940, eye 150 perceives a first image of a line having a first color and a second image of a line having a second color. When eye chart 900 with the two colored lines is moved sufficiently close to eye 150, then eye 150 is no longer able to resolve the two images of the two colored lines. Eye 150 then perceives an image of a line having a color that is an additive mixture of the first color and the second color. The point at which eye 150 changes from the state of “resolution” of the images of the two colored lines to the state of “no resolution” of the images of the two colored lines is very noticeable because of the sudden appearance of a line having a new color that occurs as a result of the additive mixture of the first color and the second color.

In an alternate embodiment of the invention, a display screen may be used in place of screen 140 or eye chart 900. FIG. 10 is a diagram illustrating display screen 1000 of the present invention. Display screen 1000 displays two colored parallel lines, 1020 and 1040. Line 1020 and line 1040 are each colored with a different color. Line 1020 and line 1040 are located with respect to each other in the same fashion as slit 120 and slit 130. The principles of operation of this alternate embodiment using display screen 1000 are the same as the principles of operation that have been previously described for the case of two separate light sources. When display screen 1000 with the two colored parallel lines, 1020 and 1040, is moved sufficiently close to eye 150, then eye 150 is no longer able to resolve the two images of the two colored parallel lines. Display screen 1000 may comprise a computer monitor, or a television cathode ray tube, or a liquid crystal display monitor, or a projection screen, or any type of screen on which an image may be displayed.

In another alternate embodiment of the invention, a display screen may be used that is capable of displaying flickering colored lines. FIG. 11 is a diagram illustrating display screen 1100 of the present invention. Display screen 1100 displays two colored parallel lines, 1120 and 1140. Line 1120 and line 1140 are each colored with a different color. Line 1120 and line 1140 are located with respect to each other in the same fashion as slit 120 and slit 130. Display screen 1100 is capable of causing colored line 1120 to flicker (i.e., rapidly turn on and off) at a first frequency. Display screen 1100 is also capable of causing colored line 1140 to flicker at the same first frequency or at a second frequency.

It is known that flicker rates greater than about sixty cycles per second (60 Hz) are not normally discernible by the human eye. The range of flicker rates that are normally discernible range from approximately five cycles per second (5 Hz) at the low end to approximately fifty cycles per second (50 Hz) at the high end.

When a person views a flickering light in which the flicker rate is increasing, at some point the person will cease to perceive any flickering. The flicker frequency at which this occurs is called the “critical flicker fusion frequency.” The critical flicker fusion frequency is related to the intensity of the light and to the color of the light (i.e., the wavelength of the light). See, generally, “Adler's Physiology of the Eye,” by Robert A. Moses, Fifth Edition, pp. 607-611, The C. V. Mosby Company, Saint Louis, 1970.

As display screen 1100 moves closer to the eye, the lack of ability of the eye to keep an image focused at close range causes the image on the retina to broaden or spread. This causes the intensity of incident light per retinal area to decrease as the image becomes unfocused and spreads out. This decrease in intensity of incident light changes the critical flicker fusion frequency.

The present invention employs the ability of the eye to not perceive the flickering of light when the flicker rate is greater than the critical flicker fusion frequency. In one advantageous embodiment of the present invention, display screen 1100 displays a first color line 1120 that flickers a first frequency and a second color line 1140 that flickers at a second frequency. As display screen 1100 is moved closer to eye 150, at some point the person perceives that the two flickering colored lines combine to produce an additive mixture that is readily discernible. By adjusting the intensity and the flicker rate of the first and second colored lines, 1120 and 1140, one can adjust flicker rate of the line that represents their combined additive mixture. The flicker rate of the line that represents the combined additive mixture may also be adjusted to a zero rate (i.e., the line does not appear to flicker).

In one advantageous embodiment of the present invention, the intensity and the first frequency of first color line 1120 and the intensity of the second frequency of second color line 1140 may be chosen so that the additive mixture of the two lines does not appear to flicker. This arrangement produces an image of the type shown in FIG. 12. FIG. 12 illustrates the appearance of three bands of light created by the combination of first flickering color line 1120 and second flickering color line 1140. First flickering color line 1120 appears as line 1220. Second flickering color line 1140 appears as line 1240. Line 1230 represents the additive mixture of first flickering color line 1120 and second flickering color line 1140. In this embodiment, line 1230 does not flicker. This is due to the fact that the flicker rate for line 1230 exceeds the critical flicker fusion frequency for the eye. The sudden appearance of a non-flickering line between two flickering lines is readily perceived by the person whose eye is being tested.

The method of testing the resolution of the eye by causing two lines to create an image of a line that flickers with a flicker rate greater than the critical flicker fusion frequency is not limited to lines having different colors. Specifically, first flickering color line 1120 that appears as line 1220 can have the same color as second flickering color line 1140 that appears as line 1240. The appearance of an image that does not appear to flicker (i.e., line 1230) can be readily perceived even if the color of line 1220 and the color of line 1240 are the same.

In another advantageous embodiment of the present invention, the intensity and the first frequency of first color line 1120 and the intensity and the second frequency of second color line 1140 may be chosen so that (1) the additive mixture of the two lines does appear to flicker, and (2) the image of first color line 1120 and the image of second color line 1140 do not appear to flicker. This arrangement produces an image of the type shown in FIG. 13. FIG. 13 illustrates the appearance of three bands of light created by the combination of first flickering color line 1120 and second flickering color line 1140. First flickering color line 1120 appears as line 1320. Second flickering color line 1140 appears as line 1340. Line 1330 represents the additive mixture of first flickering color line 1120 and second flickering color line 1140. In this embodiment, line 1330 does flicker. This is due to the fact that the flicker rate for line 1330 is less than the critical flicker fusion frequency for the eye. The sudden appearance of a flickering line between two non-flickering lines is readily perceived by the person whose eye is being tested.

The method of testing the resolution of the eye by causing two non-flickering lines to create an image of a line that flickers is not limited to lines having different colors. Specifically, first non-flickering color line 1120 that appears as line 1320 can have the same color as second non-flickering color line 1140 that appears as line 1340. The appearance of an image that appears to flicker (i.e., line 1330) can be readily perceived even if the color of line 1320 and the color of line 1340 are the same.

The advantage of using colored line inheres in the fact that the particular values of the critical flicker fusion frequency in particular cases depends upon the color of light that is employed. In an advantageous embodiment of the invention, the present invention can provide a suddenly appearing line having (1) a new color that occurs as a result of the additive mixture of the first color and the second color, and having (2) a flickering image between two non-flickering line images. Alternatively, the present invention can provide a suddenly appearing line having (1) a new color that occurs as a result of the additive mixture of the first color and the second color, and having (2) a non-flickering image between two flickering line images.

In an alternate embodiment of the present invention the first flicker frequency and the second flicker frequency have the same frequency. In another alternate embodiment of the present invention the light sources used comprise laser light sources.

The present invention therefore provides an improved system and method for testing and determining the resolving power of the eye. The present invention comprises a system and method to enable a person whose eye is being tested to easily determine a definite end point to precisely determine when the eye has ceased to resolve two individual images.

Although the present invention has been described in detail, those skilled in the art should understand that they can make various changes, substitutions and alterations herein without departing from the spirit and scope of the invention in its broadest form.

What is claimed is:

1. A method for providing a test to determine resolving power of an eye, said method comprising the steps of:
illuminating a lens of the eye with a first light source having a first color;
simultaneously illuminating the lens of the eye with a second light source having a second color; and
moving the first light source and the second light source toward the eye to a point at which the first light source color and the second light source color combine to form a third color that is an additive mixture of the first color and the second color.

2. A method as claimed in claim 1 wherein said first light source comprises light of a first color that passes through a first slit of an opaque screen and said second light source comprises light of a second color that passes through a second slit of said opaque screen.

3. A method as claimed in claim 2 wherein said first slit and said second slit each has a width of approximately two hundred microns and a height in a range from approximately five millimeters to ten millimeters, and wherein said first slit and said second slit are separated by a distance that is in a range from approximately one hundred microns to one thousand microns.

4. A method as claimed in claim 1 wherein said first light source comprises a first colored line of an eye chart illuminated by ambient light and said second light source comprises a second colored line of said eye chart illuminated by ambient light.

5. A method as claimed in claim 1 wherein said first light source comprises light of a first color from a display screen and said second light source comprises light of a second color from said display screen.

6. A method as claimed in claim 5 wherein said display screen comprises one of: a computer monitor, a television cathode ray tube, a projection screen, and a screen on which an image may be displayed.

7. A method as claimed in claim 1 wherein said first color is yellow, and wherein said second color is blue, and wherein said third color is white.

8. A method as claimed in claim 1 wherein said first color is red, and wherein said second color is green, and wherein said third color is yellow.

9. A method for providing a test to determine resolving power of an eye, said method comprising the steps of:
illuminating a lens of the eye with light from a first colored line having a first color;
simultaneously illuminating the lens of the eye with light from a second colored line having a second color; and
moving the first colored line and the second colored line toward the eye to a point at which the color of the first colored line and the color of the second colored line combine to form a line having a third color that is an additive mixture of the first color and the second color.

10. A method as claimed in claim 9 wherein said first colored line and said second colored line each has a width of approximately two hundred microns and a height in a range from approximately five millimeters to ten millimeters, and wherein said first colored line and said second colored line are separated by a distance that is in a range from approximately one hundred microns to one thousand microns.

11. A method as claimed in claim 9 wherein said first color is yellow, and wherein said second color is blue, and wherein said third color is white.

12. A method as claimed in claim 9 wherein said first color is red, and wherein said second color is green, and wherein said third color is yellow.

13. A method for providing a test to determine resolving power of an eye, said method comprising the steps of:
illuminating a lens of the eye with a first light source having a first color wherein light from said first light source flickers at a first flicker rate;
simultaneously illuminating the lens of the eye with a second light source having a second color wherein light from said second light source flickers at a second flicker rate; and
moving the first light source and the second light source toward the eye to a point at which the first light source color and the second light source color combine to form a third color that is an additive mixture of the first color and the second color.

14. A method as claimed in claim 13
wherein light from said first light source flickers at a first flicker rate that is less than a critical flicker fusion frequency for the eye;
wherein light from said second light source flickers at a second flicker rate that is less than a critical flicker fusion frequency for the eye; and
wherein light of said third color that is an additive mixture of the first color and the second color flickers at a flicker rate that is greater than a critical flicker fusion frequency for the eye.

15. A method as claimed in claim 14 wherein said first color and said second color have a same color.

16. A method as claimed in claim 14 wherein said first color is yellow, and wherein said second color is blue, and wherein said third color is white.

17. A method as claimed in claim 14 wherein said first color is red, and wherein said second color is green, and wherein said third color is yellow.

18. A method as set forth in claim 13
wherein light from said first light source flickers at a first flicker rate that is greater than a critical flicker fusion frequency for the eye;
wherein light from said second light source flickers at a second flicker rate that is greater than a critical flicker fusion frequency for the eye; and
wherein light of said third color that is an additive mixture of the first color and the second color flickers at a flicker rate that is less than a critical flicker fusion frequency for the eye.

19. A method as claimed in claim 18 wherein said first color and said second color have a same color.

20. A method as claimed in claim 18 wherein said first color is yellow, and wherein said second color is blue, and wherein said third color is white.

21. A method as claimed in claim 18 wherein said first color is red, and wherein said second color is green, and wherein said third color is yellow.

22. An apparatus for determining resolving power of an eye, said apparatus comprising:
a first light source having a first color; and
a second light source having a second color;
wherein the first light source and the second light source are capable of being moved toward the eye to a position where the eye is no longer able to resolve an image of the first light source and an image of the second light source.

23. An apparatus as claimed in claim 22 comprising a opaque screen formed having portions that define two slits through said opaque screen, wherein said first light source comprises light of a first color that passes through a first slit of said opaque screen, and wherein said second light source comprises light of a second color that passes through a second slit of said opaque screen.

24. An apparatus as claimed in claim 23 wherein said first slit and said second slit of said opaque screen each has a width of approximately two hundred microns and a height in a range from approximately five millimeters to ten millimeters, and wherein said first slit and said second slit are separated by a distance that is in a range from approximately one hundred microns to one thousand microns.

25. An apparatus as claimed in claim 22 comprising a eye chart having a first colored line and a second colored line, wherein said first light source comprises light from said first colored line illuminated by ambient light, and wherein said second light source comprises light from said second colored line illuminated by ambient light.

26. An apparatus as claimed in claim 22 comprising a display screen that displays a first colored line and a second colored line, wherein said first light source comprises light from said first colored line displayed by said display screen, and wherein said second light source comprises light from said second colored line displayed by said display screen.

27. An apparatus as claimed in claim 26 wherein said display screen comprises one of: a computer monitor, a television cathode ray tube, a projection screen, and a screen on which an image may be displayed.

28. An apparatus as claimed in claim 26 wherein said display screen is capable of displaying said first colored line at a first flicker frequency and is capable of displaying said second colored line at a second flicker frequency.

29. An apparatus as claimed in claim 28 wherein said display screen is capable of displaying said first colored line at a first flicker frequency that is less than a critical flicker fusion frequency for the eye and is capable of displaying said second colored line at a second flicker frequency that is less than a critical flicker fusion frequency for the eye.

30. An apparatus as claimed in claim 29 wherein said first flicker frequency and said second flicker frequency have a same frequency.

31. An apparatus as claimed in claim 28 wherein said display screen is capable of displaying said first colored line at a first flicker frequency that is greater than a critical flicker fusion frequency for the eye and is capable of displaying said second colored line at a second flicker frequency that is greater than a critical flicker fusion frequency for the eye.

32. An apparatus as claimed in claim 31 wherein said first flicker frequency and said second flicker frequency have a same frequency.
